(19) United States
(12) Patent Application Publication
(10) Pub. No.: US 2019/0304657 A1
(43) Pub. Date: Oct. 3, 2019

(54) ELECTRIC TRANSFORMER

(71) Applicant: LSIS CO., LTD., Anyang-si (KR)

(72) Inventors: Chang-Hwan JIN, Anyang-si (KR); Jong-Ung CHOI, Anyang-si (KR); Hyun-Ho KWON, Anyang-si (KR); Gwang-Goo KANG, Anyang-si (KR); Jin-Ho LEE, Anyang-si (KR)

(21) Appl. No.: 16/193,712

(22) Filed: Nov. 16, 2018

(30) Foreign Application Priority Data
Mar. 28, 2018 (KR) 10-2018-0035856
Mar. 28, 2018 (KR) 10-2018-0035857

(51) Int. Cl. H01F 27/12; H01F 27/40; H01F 27/32; G01N 33/28

(52) U.S. Cl. CPC H01F 27/12; G01N 33/2841; H01F 27/32; H01F 27/402

(57) ABSTRACT

An electric transformer is provided. The electric transformer includes an electric transformer main body and a heat radiating portion including a pipe configured to introduce an insulating oil into the electric transformer main body so as to absorb heat generated from the electric transformer main body, and the pipe is provided with a fixing member to which a sensing member is coupled such that the sensing member is exposed to the inside of the pipe so as to detect a dissolved gas in the insulating oil.

### CROSS-REFERENCE TO RELATED APPLICATION

Pursuant to 35 U.S.C. § 119(a), this application claims the benefit of earlier filing date and right of priority to Korean Application No. 10-2018-0035856 filed on Mar. 28, 2018, and Korean Application No. 10-2018-0035857, filed on Mar. 28, 2018, whose entire disclosures are herein incorporated by reference.

### BACKGROUND

### 1. Field of the Invention

The present disclosure relates to an electric transformer, and more particularly, to an electric transformer which may be easily coupled to a gas detection sensor to analyze a state of an insulating oil of a hydraulic transformer.

### 2. Description of Related Art

In general, an electric transformer is installed to increase or decrease a voltage of a transmission/distribution line. The electric transformer plays a large role in stably supplying electric power as an important component of an electric power system.

The electric transformer is filled with an insulating oil so as to prevent a dielectric strength of a winding provided in the electric transformer from being lowered due to moisture or dust, and to radiate heat generated from the winding by means of convection or radiation of the oil.

An electric discharge occurring within the transformer causes an abnormal phenomenon such as pyrolysis, local overheating, and an increase in temperature resulting from operation. As a result, insulating materials such as, for example, an insulating oil, an insulating paper and the like may be decomposed by heat to generate gases such as H₂, CO, C₂H₂ and C₂H₄, and the generated gases may be dissolved in the insulating oil.

A method for detecting gas contained in the insulating oil is divided into an oil valve direct connection method and an oil circulation method.

The above two methods are the same in that the insulating oil in the transformer is taken into the data acquisition device, analyzed, and then inserted into the transformer. However, in the oil valve direct connection method, the sensor is directly connected to the oil valve of the transformer to analyze the insulating oil, while in the oil circulation method, the oil is taken from the oil valve and analyzed and put back into the oil valve.

Recently, studies on a structure for mounting a gas detection sensor such that the data acquisition device analyzes a state of the insulating oil without directly taking the insulating oil have been ongoing.

### SUMMARY OF THE INVENTION

Embodiments of the present disclosure provide an electric transformer provided with a fixing member which may be easily coupled to a gas detection sensor to analyze a state of an insulating oil of a hydraulic transformer.

According to embodiments of the present disclosure, there is provided an electric transformer. The electric transformer may include an electric transformer main body and a heat radiating portion including a pipe configured to introduce an insulating oil into the electric transformer main body so as to absorb heat generated from the electric transformer main body, and the pipe is provided with a fixing member to which a sensing member is coupled such that the sensing member is exposed to the inside of the pipe to detect a dissolved gas in the insulating oil.

The fixing member may include a head portion exposed on an outer surface of the pipe and a body portion exposed to the inside of the pipe and provided with an exposure hole such that the insulating oil is in contact with the sensing member.

The head portion may be provided with an insertion hole into which the sensing member is inserted in a first direction.

The body portion may be provided with a coupling hole to which the sensing member inserted in the first direction is coupled.

An inner surface of the coupling hole is provided with a thread to which the sensing member is screw-coupled.

The fixing member may further include a blocking plate inserted and fixed to the head portion to prevent backflow of the insulating oil when the sensing member is not coupled.

The head portion may be provided with an insertion hole into which the sensing member is inserted in the first direction, the body portion may be provided with a coupling hole to which the sensing member inserted in the first direction is coupled, and the head portion may be provided a separation hole to which the blocking plate is inserted and fixed such that the insertion hole and the coupling hole are separated from each other.

The head portion may be provided with a confirmation hole to visually confirm whether or not the sensing member is coupled and the blocking plate is inserted.

The fixing member may include a head portion exposed to an outer surface of the pipe and provided with a first insertion hole into which the sensing member is inserted, a body portion exposed to the inside of the pipe and provided with a second insertion hole connected to the first insertion hole, and a blocking plate member provided at a bottom portion of the body portion and configured to separate the first and second insertion holes from each other when the sensing member is not inserted.

When the sensing member is inserted, the head portion may be provided with a screw groove to which the sensing member is coupled.

The body portion may be provided with an exposure hole through which the insulating oil passes.

The blocking plate member may include a spring provided at the bottom portion and having an elastic force, and a blocking plate disposed on the spring and configured to separate the first and second insertion holes from each other.

The blocking plate may be in contact with the sensing member and be configured to compress the spring when the sensing member is inserted.

The electric transformer according to embodiments of the present disclosure is advantageous in that a sensing member including a gas detection sensor is coupled to a fixing member provided in a hydraulic pipe at a position desired by a user or operator.

Further, since the sensing member coupled to the fixing member directly transmits a detection signal that detects a dissolved gas in the insulating oil to a data acquisition device, it is not necessary to install an additional valve or the like to detect the dissolved gas in the insulating oil, thereby reducing a manufacturing cost of the electric transformer and a space for installation.

### BRIEF DESCRIPTION OF THE DRAWINGS

FIG. 1 is a perspective view of an electric transformer according to an embodiment of the present disclosure.

FIG. 2 is a schematic cross-sectional view of an inflow pipe of an electric transformer according to a first embodiment of the present disclosure.

FIG. 3 is a perspective view of the fixing member illustrated in FIG.2.

FIG. 4 illustrates one side surface and the other side surface adjacent thereto of the fixing member illustrated in FIG. 3.

FIG. 5 is a cross-sectional view of the fixing member illustrated in FIG. 3 according to a first embodiment of the present disclosure.

FIG. 6 is a cross-sectional view of the fixing member illustrated in FIG. 3 according to a second embodiment of the present disclosure.

FIG. 7 is a schematic cross-sectional view of an inflow pipe of an electric transformer according to a second embodiment of the present disclosure.

FIG. 8 is a perspective view of the fixing member illustrated in FIG. 7.

FIG. 9 is a cross-sectional view of the fixing member illustrated in FIG. 7.

FIG. 10 illustrates a process in which a sensing member is inserted into the fixing member illustrated in FIG. 7.

### DETAILED DESCRIPTION OF EXEMPLARY EMBODIMENTS

The above-described objects, features and advantages will be described in detail with reference to the accompanying drawings, so that those skilled in the art can easily carry out a technical idea of the present disclosure. In the description of the embodiments, the detailed description of well-known related configurations or functions will be omitted when it is deemed that such description will cause ambiguous interpretation of the present disclosure. Hereinafter, preferred embodiments of the present disclosure will be described in detail with reference to the accompanying drawings. In the drawings, same reference numerals designate same or like elements.

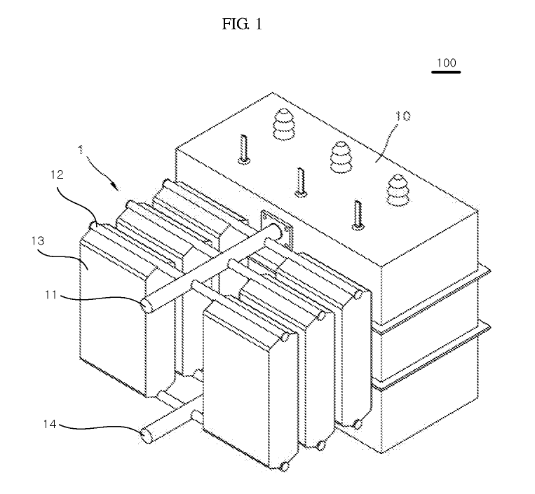

FIG. 1 is a perspective view of an electric transformer according to an embodiment of the present disclosure.

Referring to FIG. 1, the electric transformer 100 may include a heat radiator 1 and an electric transformer main body 10.

Here, the heat radiator 1 may include a first inflow pipe 11 introduced from the electric transformer main body 10, a second inflow pipe 12 branched from the first inflow pipe 11, a plurality of heat radiating plates 13 connected to a lower portion of the second inflow pipe 12 to heat-exchange the insulating oil, and a discharging pipe connected to a lower portion of the heat radiating plate 13 to discharge the heat exchanged insulating oil to the electric transformer main body 10.

The first inflow pipe 11 of the heat radiator 1 may be connected to one side of the electric transformer main body 10 in a horizontal direction, and the plurality of heat radiating plates 13 may be disposed on a lower portion of the second inflow pipe 12 branched from the first inflow pipe 11 so as to be spaced apart from each other.

The above-described forms of the first and second inflow pipes 11 and 12 and the plurality of heat radiating plates 13 are merely one embodiment for describing the electric transformer 100. The first and second inflow pipes 11 and 12 and the plurality of heat radiating plates 13 are not limited thereto, and may be configured in other forms.

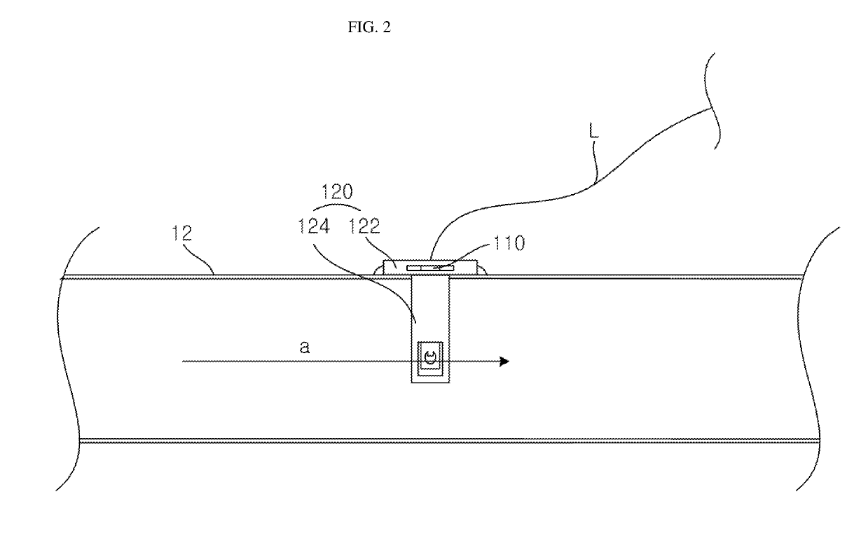

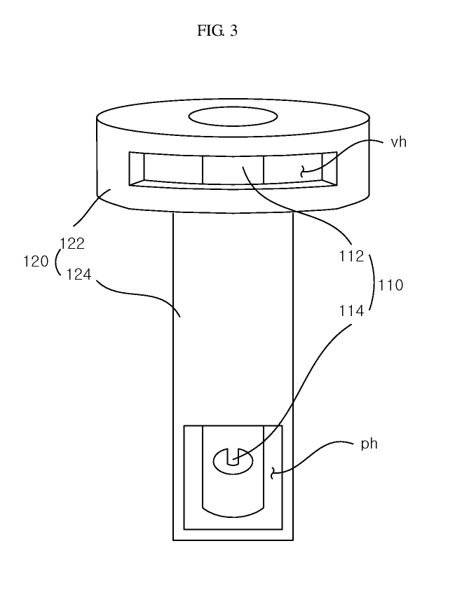

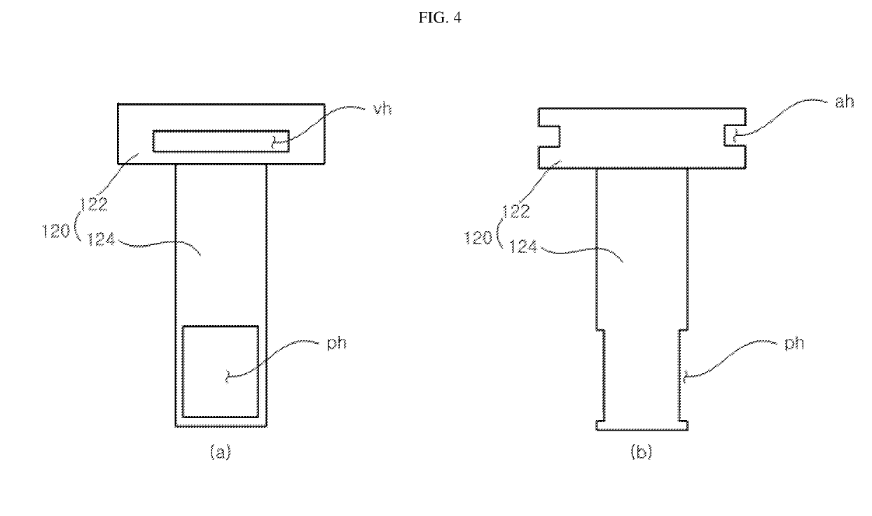

FIG. 2 is a schematic cross-sectional view of an inflow pipe of an electric transformer according to a first embodiment of the present disclosure, FIG. 3 is a perspective view of the fixing member illustrated in FIG. 2, and FIG. 4 illustrates one side surface and the other side surface adjacent thereto of the fixing member illustrated in FIG. 3.

FIG. 2 illustrates the inflow pipe as the second inflow pipe 12 among the first and second inflow pipes 11 and 12 illustrated in FIG. 1. But, the inflow pipe is not limited to the second inflow pipe, and may be the first inflow pipe 11 or a discharging pipe 14.

Referring to FIGS. 2 to 4, the second inflow pipe 12 may include a sensing member 110 and a fixing member 120.

In one embodiment, the sensing member 110 may be coupled to the fixing member 120.

The fixing member 120 may be formed integrally with the second inflow pipe 12. In an embodiment, the fixing member 120 may be inserted into a hole (not shown) provided in the second inflow pipe 12 and be coupled thereto through welding, which can be considered as one piece.

The fixing member 120 may include a head portion 122 exposed to an outer surface of the second inflow pipe 12 and welded to the outer surface of the second inflow pipe 12, and a body portion 124 provided with an exposure hole ph such that an insulating oil (not shown) is in contact with the sensing member 110.

The exposure hole ph may be formed so as not to block the flow of the insulating oil.

Here, the head portion 122 may be provided with an insertion hole (not shown) into which the sensing member 110 is inserted, and the body portion 124 may be provided with a coupling hole (not shown) to which the sensing member 110 inserted into the insertion hole is coupled.

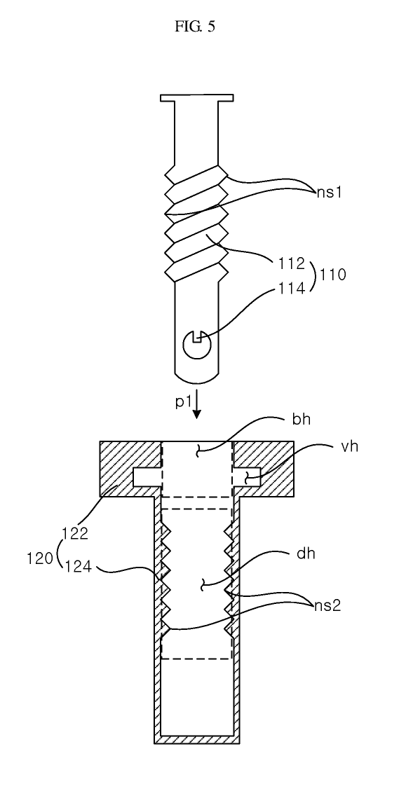

The detailed description of the insertion hole and the coupling hole will be described later with reference to FIGS. 4 and 5.

The head portion 122 may be provided with a confirmation hole vh to visually confirm whether or not the sensing member 110 is coupled. That is, the confirmation hole vh may allow the user or the operator to visually confirm whether or not the sensing member 110 is coupled. When a size of the confirmation hole vh is small, it is possible to confirm whether or not the sensing member 110 is coupled through a color of the sensing member 110.

In addition, when the sensing member 110 is not coupled, the head portion 122 may be provided with a separation hole ah into which a blocking plate (not shown) is inserted so as to separate the insertion hole and the coupling hole from each other.

The sensing member 110 may include a body portion 112 and a sensing portion 114.

The body portion 112 may have a built-in sensing portion 114 and a signal line L configured to transmit a sensor value (not shown) corresponding to a dissolved gas in the insulating oil detected by the sensing portion 114 to an external data acquisition device (not shown). Also, the sensing portion 114 may transmit the sensor value to the data acquisition device through wireless communication.

In an embodiment, the data acquisition device may acquire and store the sensor value for the dissolved gas in the insulating oil.

The data acquisition device may monitor a state of the insulating oil flowing in the second inflow pipe 12 as the signal line L is connected.

The body portion 112 may be formed to have a narrower width than the exposure hole ph so as to make the flow of the insulating oil smooth.

Here, the sensing portion 114 may detect the dissolved gas in the insulating oil and a temperature of the insulating oil, but is not limited thereto.

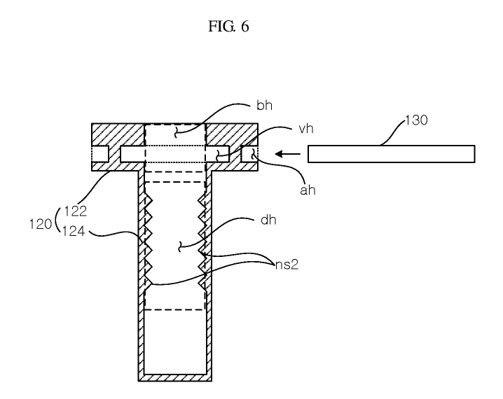

FIG. 5 is a cross-sectional view of the fixing member illustrated in FIG. 3 according to a first embodiment of the present disclosure, and FIG. 6 is a cross-sectional view of the fixing member illustrated in FIG. 3 according to a second embodiment of the present disclosure.

First, FIG. 5 illustrates that the sensing member 110 is inserted into the fixing member 120.

Referring to FIG. 5, the head portion 122 of the fixing member 120 may be provided with an insertion hole bh into which the sensing member 110 is inserted in a first direction p1, and the body portion 124 of the fixing member 120 may be provided with a coupling hole dh to which the sensing member 110 inserted into the insertion hole bh is coupled.

Here, an inner surface of the coupling hole dh may be provided with a thread ns2, and thus may be coupled to the body portion 112 provided with a thread ns1.

That is, the sensing member 110 may be rotated and coupled to the fixing member 120 for screw-coupling with the fixing member 120. Then, when the sensing member 110 is completely coupled to the fixing member 120, the sensing portion 114 may be exposed to the exposure hole ph.

As described above, the fixing member 120 may be screw-coupled and fixed to the sensing member 110, thereby preventing deviation of the sensing member 120 caused by internal pressure of the second inflow pipe 12.

FIG. 6 illustrates that the sensing member 110 is not coupled to the fixing member 120.

That is, referring to FIG. 6, when the sensing member 110 is not coupled to the fixing member 120 by the user or operator, the fixing member 120 may be provided with a separation hole ah to which an additional blocking plate 130 is inserted and fixed by the user or operator.

Here, the blocking plate 130 may separate the insertion hole bh of the head portion 122 and the coupling hole dh of the body portion 124 from each other, thereby preventing the insulating oil flowing in the second inflow pipe 12 from flowing back to the head portion 122.

When the sensing member 110 is coupled to the fixing member 120, the thread ns2 of the body portion 124 and the thread ns1 of the body portion 114 of the sensing member 110 may be screw-coupled to each other to prevent backflow of the insulating oil.

However, when the sensing member 110 is not deviated from or coupled to the fixing member 120, the insulating oil may flow back to the outside of the second inflow pipe 12 by the internal pressure. Thus, the blocking plate 130 may be coupled to the separation hole ah provided in the head portion 122 to prevent backflow of the insulating oil.

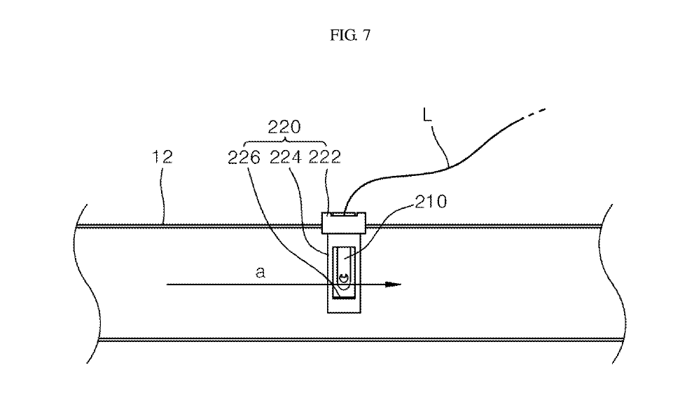

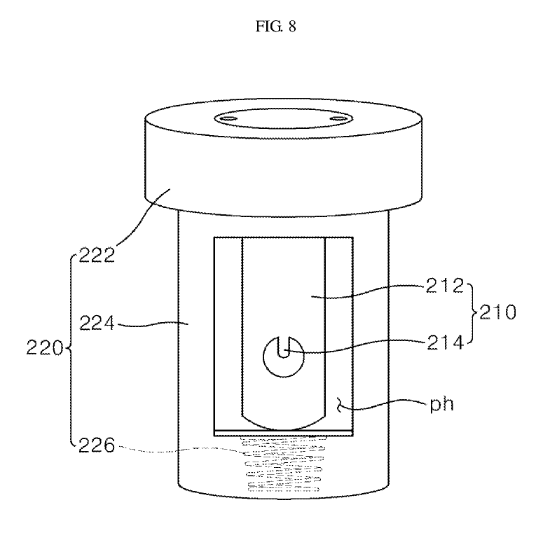

FIG. 7 is a schematic cross-sectional view of an inflow pipe of an electric transformer according to a second embodiment of the present disclosure, and FIG. 8 is a perspective view of the fixing member illustrated in FIG. 7.

FIG. 7 illustrates the inflow pipe as the second inflow pipe 12 among the first and second inflow pipes 11 and 12 illustrated in FIG. 1. But, the inflow pipe is not limited to the second inflow pipe, and may be the first inflow pipe 11 or the discharging pipe 14.

Referring to FIGS. 7 and 8, the second inflow pipe 12 may include a sensing member 210 and a fixing member 220.

In one embodiment, the sensing member 210 may be inserted into the fixing member 220.

The fixing member 220 may be formed integrally with the second inflow pipe 12. In an embodiment, the fixing member 220 may be inserted into a hole (not shown) provided in the second inflow pipe 12, and may be coupled thereto through welding, which can be considered as one piece.

The fixing member 220 may include a head portion 222 exposed to the outer surface of the second inflow pipe 12 and welded to the outer surface of the second inflow pipe 12, a body portion 224 exposed to the inside of the second inflow pipe and provided with an exposure hole ph such that an insulating oil (not shown) is in contact with the sensing member 210, a blocking plate member 226 provided at a bottom portion of the body portion 224 and configured to move in accordance with insertion of the sensing member 110.

The exposure hole ph may be formed so as not to block the flow of the insulating oil.

Here, the head portion 222 may be provided with a first insertion hole (not shown) into which the sensing member 210 is inserted.

Further, the body portion 224 may be provided with a second insertion hole (not shown) which is connected to the sensing member 210 inserted into the first insertion hole.

Here, the body portion 224 may be formed in a cylindrical structure in which the bottom portion exists, and provided with an empty space therein, that is, the second insertion hole by coupling with the head portion 222.

The blocking plate member 226 may include a spring (not shown) and a blocking plate (not shown).

Here, the spring may have an elastic force such that it is compressed and relaxed, and the blocking plate is movable up and down depending on whether or not sensing member 210 is inserted.

The blocking plate may separate or connect the first insertion hole of the head portion 222 and the second insertion hole of the body portion 226 from or to each other.

The detailed description of the spring, the blocking plate, and the first and second insertion holes will be described later.

The sensing member 210 may include a body portion 212 and a sensing portion 214.

The body portion 212 may have a built-in sensing portion 214 and a signal line L configured to transmit a sensor value (not shown) corresponding to a dissolved gas in the insulating oil detected by the sensing portion 214 to an external data acquisition device (not shown). Also, the sensing portion 114 may transmit the sensor value to the data acquisition device through wireless communication.

In an embodiment, the data acquisition device may acquire and store the sensor value for the dissolved gas in the insulating oil.

The data acquisition device may monitor a state of the insulating oil flowing in the second inflow pipe 12 as the signal line L is connected.

The body portion 212 may be formed to have a narrower width than the exposure hole ph so as to make the flow of the insulating oil smooth.

Further, an upper portion of the body portion 212 may be provided with a screw hole (not shown) for screw-coupling with the head portion 222 of the fixing member 220.

Here, the sensing portion 214 may detect the dissolved gas in the insulating oil and a temperature of the insulating oil, but is not limited thereto.

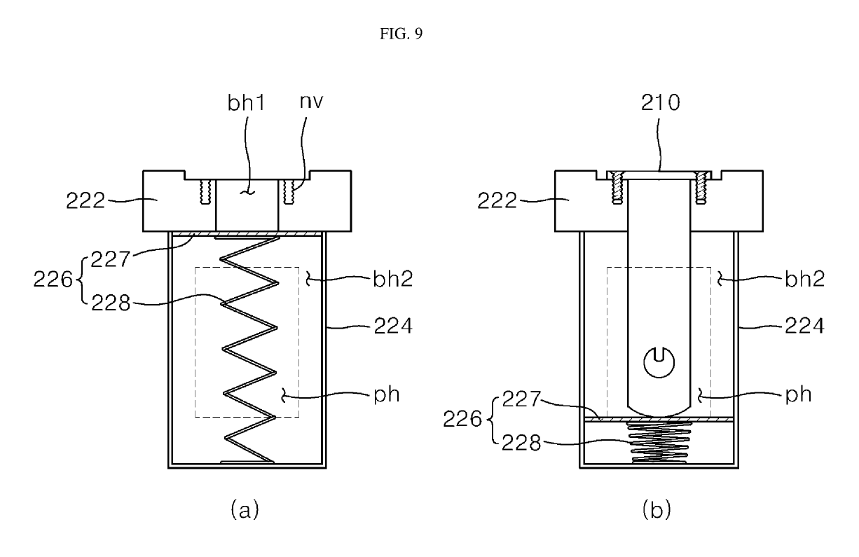

FIG. 9 is a cross-sectional view of the fixing member illustrated in FIG. 7.

(a) in FIG. 9 illustrates a cross-section of the fixing member 222 into which the sensing member 210 is not inserted, and (b) in FIG. 8 illustrates a cross-section of the fixing member 220 into which the sensing member 210 is inserted.

Referring to (a) in FIG. 9, the fixing member 220 may include a head portion 222, a body portion 224, and a blocking plate member 226.

The head portion 222 may be provided with a first insertion hole bh1 into which the sensing member 210 is inserted and a screw groove nv to allow the inserted sensing member 210 to be screw-coupled to the fixing member 220.

The sensing member 210 may provided with a thread to which the screw groove nv provided in the first insertion hole bh1 is screw-coupled.

Here, the width of the first insertion hole bh1 may be 1 to 1.2 times that of the sensing member 210.

When the width of the first insertion hole bh1 is 1.2 times larger than that of the sensing member 210, a distance between the first insertion hole bh1 and the sensing member 210 inserted into the first insertion hole bh1 may be widened, and thus the sensing member 210 may not be properly screw-coupled to the fixing member 210. As a result, it may be difficult to prevent backflow of the insulating oil.

In addition, the body portion 224 may be formed in a cylindrical structure with an upper portion thereof closed by the head portion 222 and a bottom portion thereof closed.

Here, when the sensing member 210 is inserted, the body portion 224 may be provided with an exposure hole ph through which the insulating oil flows at a side thereof such that the sensing member 210 detects the dissolved gas in the insulating oil.

The body portion 224 may have a cylindrical structure, and be provided with a second insertion hole bh2 which is connected to the first insertion hole bh1 and into which the sensing member 210 is inserted.

In one embodiment, the second insertion hole bh2 may be formed as an insertion groove, or may be an empty space of the body portion 224 having a cylindrical structure.

Here, the width of the second insertion hole bh2 may be 1 to 1.2 times that of the first insertion hole bh1.

That is, when the width of the second insertion hole bh2 is 1.5 times less than that of the first insertion hole bh1, it may be difficult to prevent backflow of the insulating oil in a state in which the first and second insertion holes bh1 and bh2 are separated from each other by the blocking plate member 226. When the width of the second insertion hole bh2 is twice larger than that of the first insertion hole bh1, it may be easy to prevent backflow of the insulating oil in a state in which the first and second insertion holes bh1 and bh2 are separated from each other by the blocking plate member 226, but effectiveness may be lowered.

The blocking plate member 226 may include a blocking plate 227 and a spring 228.

The blocking plate 227 may separate or connect the first and second insertion holes bh1 and bh2 from or to each other.

That is, when the sensing member 210 is not inserted, the blocking plate 227 may separate the first and second insertion holes bh1 and bh2 from each other to prevent the insulating oil from flowing back to the head portion 222 through the body portion 224.

Here, the width of the blocking plate 227 may be 1.3 to 1.8 times that of the first insertion hole bh1, and may be 0.7 to 0.9 times that of the second insertion hole bh2.

Referring to (b) in FIG. 9, the fixing member 220 may include a head portion 222, a body portion 224, and a blocking plate member 226.

The head portion 222 and the body portion 224 each have the same configuration as those described with reference to (a) in FIG. 9, and thus the description thereof will be omitted.

When the insertion of the sensing member 210 is completed, the blocking plate 226 may be lowered to compress the spring 228.

The sensing member 210 may detect the dissolved gas in the insulating oil that flows through the exposure hole ph of the body portion 224.

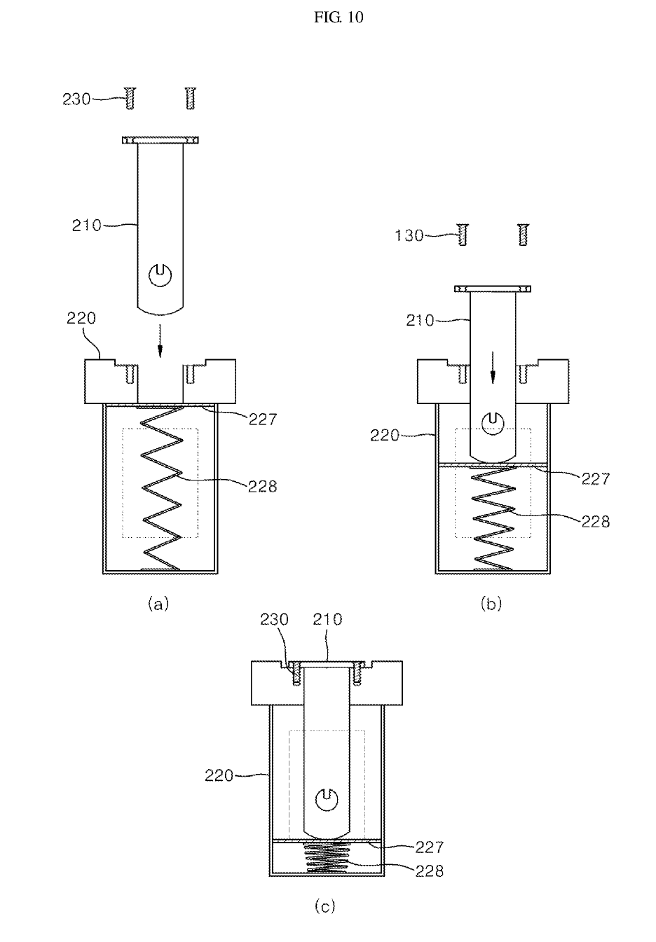

FIG. 10 illustrates a process in which a sensing member is inserted into the fixing member illustrated in FIG. 7.

(a), (b) and (c) in FIG. 10 each illustrate a process in which the sensing member 210 is inserted into the fixing member 220.

That is, (a) in FIG. 10 illustrates the fixing member 220 to be installed in the second inflow pipe 12 in a state in which the sensing member 210 is not inserted, and the fixing member of (a) in FIG. 10 may be in the same state as the fixing member of (a) in FIG. 9

(a) in FIG. 10 may illustrate a process before the sensing member 210 is inserted such that the user or operator detects the dissolved gas in the insulating oil.

(b) in FIG. 10 illustrates a process in which the sensing member 210 is inserted into the fixing member 220.

That is, (b) in FIG. 10 illustrates that the blocking plate member 226 may be brought into contact with the sensing member 210 when the sensing member 210 is inserted, and may be lowered to correspond to a degree of insertion of the sensing member 210.

Here, the blocking plate member 226 may indicate a progress in which the spring 228 is compressed as the blocking plate 227 is lowered.

(c) in FIG. 10 illustrates a state in which the sensing member 210 is completely inserted into the fixing member 220, and the screw hole nh provided in the sensing member 210 is coupled to the screw groove nv provided in the fixing member 220 by a screw 230.

FIG. 10 illustrates a process in which the sensing member 210 is inserted into the fixing member 220. However, when the inserted sensing member 210 is taken out, it can be interpreted that the processes of (a), (b), and (c) in FIG. 10 are performed in a reverse order.

The present disclosure described as above is not limited by the embodiments described herein and accompanying drawings. It should be apparent to those skilled in the art that various substitutions, changes and modifications which are not exemplified herein but are still within the spirit and scope of the present disclosure may be made.

What is claimed is:

1. An electric transformer, comprising:
an electric transformer main body; and
a heat radiating portion including a pipe configured to introduce an insulating oil into the electric transformer main body so as to absorb heat generated from the electric transformer main body, and
wherein the pipe is provided with a fixing member to which a sensing member is coupled such that the sensing member is exposed to the inside of the pipe so as to detect a dissolved gas in the insulating oil.

2. The electric transformer of claim 1, wherein
the fixing member comprises:
a head portion exposed to an outer surface of the pipe; and
a body portion exposed to the inside of the pipe and provided with a exposure hole such that the insulating oil is in contact with the sensing member.

3. The electric transformer of claim 2, wherein
the head portion further comprises:
an insertion hole into which the sensing member is inserted in a first direction.

4. The electric transformer of claim 3, wherein
the body portion further comprises:
a coupling hole to which the sensing member inserted in the first direction is coupled.

5. The electric transformer of claim 4, wherein
an inner surface of the coupling hole is provided with a thread to which the sensing member is screw-coupled.

6. The electric transformer of claim 2, wherein
the fixing member further comprises:
a blocking plate inserted and fixed to the head portion to prevent backflow of the insulating oil when the sensing member is not coupled.

7. The electric transformer of claim 6, wherein
the head portion further comprises an insertion hole into which the sensing member is inserted in a first direction,
the body portion further comprises a coupling hole to which the sensing member inserted in the first direction is coupled, and
the head portion further comprises a separation hole to which the blocking plate is inserted and fixed such that the insertion hole and the coupling hole are separated from each other.

8. The electric transformer of claim 6, wherein
the head portion further comprises:
a confirmation hole to visually confirm whether or not the sensing member is coupled and the blocking plate is inserted.

9. The electric transformer of claim 1, wherein
the fixing member comprises:
a head portion exposed to an outer surface of the pipe and provided with a first insertion hole into which the sensing member is inserted;
a body portion exposed to the inside of the pipe and provided with a second insertion hole which is connected to the first insertion hole; and
a blocking plate member provided at a bottom portion of the body portion and configured to separate the first and second insertion holes from each other when the sensing member is not inserted.

10. The electric transformer of claim 9, wherein
the head portion further comprises:
a screw groove to which the sensing member is screw-coupled when the sensing member is inserted.

11. The electric transformer of claim 9, wherein
the body portion further comprises:
an exposure hole through which the insulating oil passes.

12. The electric transformer of claim 9, wherein
the blocking plate member comprises:
a spring provided at the bottom portion and having an elastic force; and
a blocking plate disposed on the spring and configured to separate the first and second insertion holes from each other.

13. The electric transformer of claim 12, wherein
the blocking plate is in contact with the sensing member and configured to compress the spring when the sensing member is inserted.
